(19) United States
(12) Patent Application Publication
(10) Pub. No.: US 2011/0313176 A1
(43) Pub. Date: Dec. 22, 2011

(54) PROCESSES FOR PREPARING HIGHLY PURE ROTIGOTINE OR A PHARMACEUTICALLY ACCEPTABLE SALT THEREOF

(75) Inventors: Mayur Devjibhai Khunt, Gujarat (IN); Shrikant Varma, Maharashtra (IN); Nilesh Sudhir Patil, Maharashtra (IN); Haushabhau Shivaji Pagire, Maharashtra (IN); Nitin Sharadchandra Pradhan, Maharashtra (IN)

(73) Assignee: ACTAVIS GROUP PTC EHF, Hafnarfjordur (IS)

(21) Appl. No.: 13/141,151

(22) PCT Filed: Dec. 22, 2009
(86) PCT No.: PCT/IB2009/007975

§ 371 Date: Sep. 13, 2011

(30) Foreign Application Priority Data
Dec. 26, 2008 (IN) 3286/CHE/2008
Jan. 29, 2009 (IN) 200/CHE/2009

(51) Int. Cl. C07D 333/20

(52) U.S. Cl. 549/75

(57) ABSTRACT

Provided herein are convenient, industrially advantageous and environmentally friendly processes for the preparation of (−)-(S)-5-hydroxy-2-[N-n-propyl-N-2-(2-thienyl)ethylamino]tetralin (rotigotine) or a pharmaceutically acceptable salt thereof. Provided further herein is a highly pure rotigotine or a pharmaceutically acceptable salt thereof substantially free of impurities, processes for the preparation thereof, and pharmaceutical compositions comprising highly pure rotigotine or a pharmaceutically acceptable salt thereof substantially free of impurities.

### CROSS REFERENCE TO RELATED APPLICATION

This application claims the benefit of priority to Indian provisional application Nos. 3286/CHE/2008, filed on Dec. 26, 2008; and 200/CHE/2009, filed on Jan. 29, 2009; which are incorporated herein by reference in their entirety.

### FIELD OF THE DISCLOSURE

The present disclosure relates to novel processes for the preparation of (−)-(S)-5-hydroxy-2-[N-n-propyl-N-2-(2-thienyl)ethylamino]tetralin (Rotigotine) or a pharmaceutically acceptable salt thereof, in high yield and purity. Disclosed further herein is a highly pure rotigotine or a pharmaceutically acceptable salt thereof substantially free of impurities, processes for the preparation thereof, and pharmaceutical compositions comprising highly pure rotigotine or a pharmaceutically acceptable salt thereof substantially free of impurities.

### BACKGROUND

Rotigotine, also known as (S)-rotigotine, chemically named (−)-(S)-5-Hydroxy-2-[N-n-propyl-N-2-(2-thienyl)ethylamino]tetralin, is a non-ergolinic dopamine agonist and useful in the treatment of the signs and symptoms of early-stage idiopathic Parkinson's disease. Rotigotine is represented by the following structural formula I:

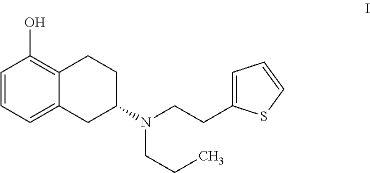

U.S. Pat. No. 4,564,628 discloses a variety of substituted 2-aminotetralin derivatives, processes for their preparation, pharmaceutical compositions and method of use thereof. These compounds are useful as dopamine agonists and, in particular, dopamine D-2 receptor agonists for the treatment of disorders of the central nervous, cardiovascular and endocrine systems such as Parkinson's disease and related disorders, hypertension and hyperprolactinemia. In particular, the compounds are useful in the treatment of glaucoma in mammals. Among them, racemic rotigotine, 5-Hydroxy-2-[N-n-propyl-N-2-(2-thienyl)ethylamino]tetralin, is a non-ergoline D₃/D₂/D₁ dopamine agonist for the treatment of Parkinson's disease. Racemic rotigotine is represented by the following structural formula:

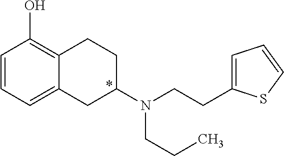

The structural formula of racemic rotigotine contains one chiral centre (the asterisk designates the chiral centre) and therefore can be resolved into its (−) and (+) isomers (enantiomers). Various processes for the preparation of 5-hydroxy-2-[N-n-propyl-N-2-(2-thienyl)ethylamino]tetralin (racemic rotigotine) and related compounds are disclosed in U.S. Pat. Nos. 4,564,628; 4,657,925; 4,885,308; 4,968,837; and 6,372,920; and European Patent No. 168505.

U.S. Pat. No. 4,564,628 (hereinafter referred to as the '628 patent) describes three synthetic routes for preparing racemic rotigotine and its hydrochloride salt. According to first synthetic process, racemic rotigotine is prepared by the reaction of 5-methoxy-2-tetralon with β-(2-thienyl)ethylamine in the presence of p-toluenesulfonic acid, followed by reduction of the resulting intermediate with sodium cyanoborohydride to produce 5-methoxy-2-[N-2-(2-thienyl)ethylamino]tetralin, which is then acylated with propionyl chloride in the presence of triethylamine in a suitable solvent to produce N-(5-methoxy-2-tetralenyl)-N-[2-(2-thienyl)ethyl)]propanamide, which is then reduced with lithium aluminum hydride to produce 5-methoxy-2-[N-n-propyl-N-2-(2-thienyl)ethylamino]tetralin, followed by demethylation with boron tribromide, and then treatment with hydrochloric acid to produce racemic rotigotine hydrochloride.

According to second synthetic process as described in the '628 patent, racemic rotigotine is prepared by the reaction of 5-methoxy-2-(N-propylamino)tetralin with 2-thiophene acetic acid in the presence of borane trimethylamine complex in xylene, or with 2-thienylacetyl chloride and lithium aluminum hydride, to produce 5-methoxy-2-[N-n-propyl-N-2-(2-thienyl)ethylamino]tetralin, which is then demethylated with boron tribromide, followed by treatment with hydrochloric acid, to produce racemic rotigotine hydrochloride. The starting material 5-methoxy-2-(N-propylamino)tetralin is prepared by reaction of 5-methoxy-2-tetralon with 3-propylamine in acetic acid followed by reduction of the resulting intermediate with H₂/PtO₂ to produce 5-methoxy-2-(N-propylamino)tetralin.

U.S. Pat. No. 4,657,925 describes both (−)-enantiomer and (+)-enantiomer of rotigotine, of which the levo (−) isomer is reported to be 140 times more potent than the (+)-isomer when used in therapy treatment.

U.S. Pat. No. 4,885,308 discloses a process for preparing the two optical isomers of rotigotine by resolving racemic 2-(N-n-propylamino)-5-methoxytetralin to its two enantiomers using an appropriate optical isomer of 4-(2-chlorophenyl)-5,5-dimethyl-2-hydroxy-1,3,2-dioxaphosphorinane-2-oxide, and then converting each enantiomer to (−) and (+)-enantiomers of rotigotine, using the processes disclosed in the '628 patent.

U.S. Pat. No. 6,372,920 (hereinafter referred to as the '920 patent) describes a process for preparing optically active and racemic nitrogen-substituted 2-aminotetralins including rotigotine. As per the process described in the '920 patent, (−)-(S)-5-hydroxy-2-[N-n-propyl-N-2-(2-thienyl)ethylamino]tetralin (rotigotine) is prepared by the reaction of (−)-5-hydroxy-N-n-propyl-2-aminotetralin with 2-(2-thienyl)ethanol toluenesulfonate in the presence of less than about 1.9-fold molar excess of an alkali metal carbonate or an alkali metal bicarbonate with respect to the amine starting material.

Drugs of the Future 1993, 18(11), 1005-1008 discloses a process for preparing rotigotine comprising methylation of 1,6-dihydroxynaphthalene with dimethyl sulfate to give 1,6-dimethoxynaphthalene, which is converted to 5-methoxy-2-tetralone by reduction with sodium in ethanol, which is then reductively aminated with propylamine to produce racemic 5-methoxy-2-N-propyl-aminotetralin, from which the (−)-enantiomer is obtained by fractional crystallization of the dibenzoyl-L-tartaric acid salt followed by demethylation with aqueous hydrobromic acid to afford (−)-(S)-5-hydroxy-2-N-propyl-aminotetralin, which is reductively alkylated with thienylacetic acid in the presence of trimethylaminoborane to produce (−)-(S)-5-hydroxy-2-[N-n-propyl-N-2-(2-thienyl)ethylamino]tetralin, which is then treated with anhydrous hydrogen chloride in ether to provide rotigotine hydrochloride.

Rotigotine obtained by the process described in the aforementioned prior art does not have satisfactory purity. Unacceptable amounts of impurities are formed along with rotigotine. The yield of rotigotine obtained is also poor and the processes involve column chromatographic purifications. Methods involving column chromatographic purifications are generally undesirable for large-scale operations, thereby making the process commercially unfeasible.

However, the prior art methods for preparing rotigotine require the use of either boron tribromide or hydrobromic acid in the demethylation reaction, which are highly corrosive and a possible tumor promoter, difficult to handle, and toxic reagents. Boron tribromide is very volatile and fumes in air because it reacts vigorously with water to form boric acid and hydrogen bromide. Chronic exposure may lead to liver or kidney damage. The use of boron tribromide and hydrobromic acid are not advisable for scale up operations.

Moreover, the prior art methods for preparing rotigotine involve the use of catalytic hydrogenation catalysts like PtO₂ catalysts for the reduction of the compounds having keto groups. However, a high cost of the catalysts and/or the necessity of using equipment suitable for operation under pressure of hydrogen, extra purification steps to obtain the final product, multiple crystallizations, and the use of explosive reagents are disadvantages of the processes used in the '628 patent.

Based on the aforementioned drawbacks, the prior art processes may be unsuitable for the preparation of rotigotine in commercial scale operations.

A need remains for an improved and commercially viable process of preparing a highly pure rotigotine or a pharmaceutically acceptable salt thereof, preferably rotigotine hydrochloride, to resolve the problems associated with the processes described in the prior art, and that will be suitable for large-scale preparation. Desirable process properties include less hazardous, environmentally friendly and easy to handle reagents, reduced cost, greater simplicity, increased purity, and increased yield of the product, thereby enabling the production of rotigotine and its pharmaceutically acceptable acid addition salts in high purity and in high yield.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in rotigotine or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure rotigotine or a pharmaceutically acceptable salt thereof substantially free of impurities, as well as processes for preparing thereof.

### SUMMARY

In one aspect, provided herein are efficient, industrially advantageous and environmentally friendly processes for the preparation of (−)-(S)-5-Hydroxy-2-[N-n-propyl-N-2-(2-thienyl)ethylamino]tetralin (Rotigotine) of formula I or a pharmaceutically acceptable salt thereof in high yield and with high chemical and enantiomeric purity. Moreover, the reagents used in the processes disclosed herein are non-hazardous and easy to handle at a commercial scale, and also allow reduced reaction times. The processes avoid the tedious and cumbersome procedures of the prior processes and are convenient to operate on a commercial scale.

In another aspect, provided herein is a highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities.

In yet another aspect, encompassed herein is a process for preparing the highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities.

In still another aspect, provided herein is a pharmaceutical composition comprising highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities made by the process disclosed herein, and one or more pharmaceutically acceptable excipients.

In still further aspect, encompassed is a process for preparing a pharmaceutical formulation comprising combining highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities with one or more pharmaceutically acceptable excipients.

In another aspect, the highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities disclosed herein for use in the pharmaceutical compositions has a 90 volume-percent of the particles (D₉₀) of less than or equal to about 400 microns, specifically less than or equal to about 300 microns, more specifically less than or equal to about 100 microns, still more specifically less than or equal to about 60 microns, and most specifically less than or equal to about 15 microns.

### DETAILED DESCRIPTION

The present inventors have fount that rotigotine obtained by the processes described in the prior art does not have satisfactory purity due to the formation of unacceptable amounts of impurities along with rotigotine. Among them, there are seven impurities identified at 0.79, 0.92, 1.12, 1.51, 1.59, 1.64 and 1.79±0.01 RRt's (hereinafter referred to as the ‘0.79 RRt’ impurity, ‘0.92 RRt’ impurity, ‘1.12 RRt’ impurity, ‘1.51 RRt’ impurity, ‘1.59 RRt’ impurity, ‘1.64 RRt’ impurity and ‘1.79 RRt’ impurity, collectively referred to as the ‘single maximum unknown impurities’), whose presence was observed in rotigotine.

Regarding the specific RRt values of impurities disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.01.

Thus there is a need for a method for determining the level of impurities in rotigotine samples and removing the impurities.

Extensive experimentation was carried out by the present inventors to reduce the level of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities in rotigotine. As a result, it has been found that the ‘0.79 RRt’, ‘0.92 RRt’, ‘1.12 RRt’, ‘1.51 RRt’, ‘1.59 RRt’, ‘1.64 RRt’ and ‘1.79 RRt’ impurities formed in the preparation of the rotigotine can be reduced or completely removed by the purification process disclosed herein.

According to one aspect, there is provided a process for the preparation of (−)-(S)-5-hydroxy-2-[N-n-propyl-N-2-(2-thienyl)ethylamino]tetralin (Rotigotine) of formula I:

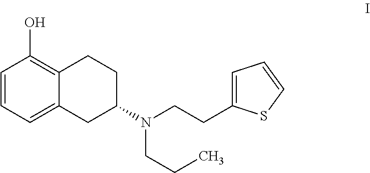

or a pharmaceutically acceptable acid addition salt thereof, comprising:
a) reacting (S)-2-amino-5-methoxytetraline of formula IV:

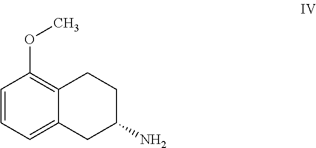

or an acid addition salt thereof with 2-(2-thienyl)-ethyl para-toluenesulfonate of formula V:

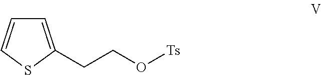

in the presence of a base in a first solvent to produce (−)-(S)-5-methoxy-2-[N-2-(2-thienyl)ethylamino]tetralin of formula III:

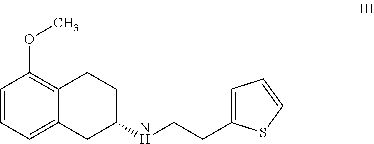

or an acid addition salt thereof;
b) reacting the compound of formula III or an acid addition salt thereof with propionic acid or propionyl halide in the presence of a reducing agent in a second solvent to produce (−)-(S)-5-methoxy-2-[N-n-propyl-N-2-(2-thienyl)ethylamino]tetralin of formula II:

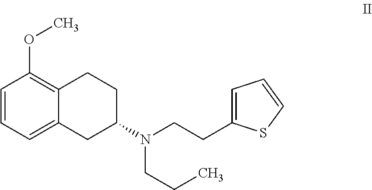

or an acid addition salt thereof;
c) demethylating the compound of formula II with a Lewis acid in the presence of thiourea in a third solvent to produce rotigotine of formula I or a pharmaceutically acceptable acid addition salt thereof; and
d) optionally, purifying the rotigotine pharmaceutically acceptable acid addition salt obtained in step-(c) with a fourth solvent to produce highly pure rotigotine pharmaceutically acceptable acid addition salt and then converting the purified rotigotine pharmaceutically acceptable acid addition salt into highly pure rotigotine free base.

The term “highly pure rotigotine or a pharmaceutically acceptable acid addition salt thereof” as used herein refers to the rotigotine or a pharmaceutically acceptable acid addition salt thereof having total purity of greater than about 99%, specifically greater than about 99.5%, more specifically greater than about 99.8%, and most specifically greater than about 99.9% (measured by HPLC).

Exemplary first solvents used in step-(a) include, but are not limited to, water, an alcohol, a chlorinated hydrocarbon, a hydrocarbon, a nitrile, an ester, an ether, a polar aprotic solvent, and mixtures thereof. In one embodiment, the reaction in step-(a) is carried out in a biphasic mixture of a hydrocarbon solvent with water. The term solvent also includes mixture of solvents.

Specifically, the first solvent is selected from the group consisting of water, methanol, ethanol, n-propanol, isopropanol, n-butanol, isobutanol, tert-butanol, amyl alcohol, hexanol, acetonitrile, ethyl acetate, methyl acetate, isopropyl acetate, tert-butyl methyl acetate, ethyl formate, acetonitrile, dichloromethane, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically the first solvent is selected from the group consisting of acetonitrile, dimethyformamide, dimethylsulfoxide, tetrahydrofuran, toluene, dichloromethane, acetone, and mixtures thereof; and a most specific first solvent is acetonitrile.

In one embodiment, the base used in step-(a) is an organic or inorganic base. Specific organic bases are triethylamine, tributylamine, diisopropylethylamine, diethylamine, tert-butyl amine, N-methylmorpholine, pyridine, 4-(N,N-dimethylamino)pyridine, and mixtures thereof. Exemplary inorganic bases include, but are not limited to, ammonia; hydroxides, alkoxides, carbonates and bicarbonates of alkali or alkaline earth metals. Specific inorganic bases are ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide, potassium tert-butoxide, and mixtures thereof; and more specifically sodium hydroxide, potassium hydroxide, sodium carbonate and potassium carbonate.

In another embodiment, the base in step-(a) is used in an amount of about 1 to 4 equivalents, specifically about 1.5 to 2 equivalents, with respect to the (S)-2-amino-5-methoxytetraline of formula IV in order to ensure a proper course of the reaction.

Advantageously, the compound of formula IV can be used in the form of an acid addition salt. Preferable acid addition salts of the compound of formula IV are hydrochloride, hydrobromide, and most preferably the hydrochloride salt.

In one embodiment, the reaction in step-(a) is carried out at a temperature of about 0° C. to the reflux temperature of the solvent used, specifically at a temperature of about 25° C. to the reflux temperature of the solvent, and more specifically at the reflux temperature of the solvent. In another embodiment, the reaction is carried out for at least 4 hours, specifically for about 5 hours to about 20 hours, and more specifically for about 14 hours to about 16 hours.

As used herein, “reflux temperature” means the temperature at which the solvent or solvent system refluxes or boils at atmospheric pressure.

In another embodiment, the reaction mass containing the (−)-(S)-5-methoxy-2-[N-2-(2-thienyl)ethylamino]tetralin of formula III obtained in step-(a) may be subjected to usual work up such as a filtration, a washing, an extractions, an evaporation, or a combination thereof, and followed by converting into its acid addition salt by reacting with an acid in a suitable solvent.

The acid addition salts of the compound of formula III are derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, and tartaric acid. A specific acid addition salt of the compound of formula III is the hydrochloride salt.

In another embodiment, the (−)-(S)-5-methoxy-2-[N-2-(2-thienyl)ethylamino]tetralin of formula III formed in step-(a) is isolated as a solid, preferably in the form of its hydrochloride salt, from a suitable organic solvent by conventional methods such as such as cooling, seeding, partial removal of the solvent from the solution, by adding an anti-solvent to the solution, evaporation, vacuum drying, spray drying, freeze drying, or a combination thereof.

The organic solvent used for isolating the compound of formula III is selected from the group consisting of methanol, ethanol, n-propanol, isopropanol, n-butanol, isobutanol, tert-butanol, amyl alcohol, hexanol, acetonitrile, ethyl acetate, methyl acetate, isopropyl acetate, tert-butyl methyl acetate, ethyl formate, acetonitrile, acetone, methyl isobutyl ketone, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof. A most specific organic solvent is ethyl acetate.

In yet another embodiment, the reaction mass containing the (−)-(S)-5-methoxy-2-[N-2-(2-thienyl)ethylamino]tetralin of formula III obtained after completion of the reaction is concentrated to yield a residue. Water and an organic solvent, preferably ethyl acetate, are added to the residue followed by the addition of concentrated hydrochloric acid to get pH acidic, preferably 1 to 2. The obtained biphasic acidic mixture is heated to reflux over a period of about 20 minutes to 1 hour. The resulting slurry is cooled to 0° C. to 30° C., specifically 0° C. to 5° C. prior to filtration. The precipitated product is filtered and optionally washed with ethyl acetate, preferably chilled ethyl acetate followed by drying the material in air oven at 50° C. to 55° C. for about 4 hours to about 8 hours.

In one embodiment, the propionyl halide used in step-(b) is propionyl chloride or propionyl bromide.

In another embodiment, the reducing agent used in step-(b) includes, but is not limited to, a metal hydride such as sodium borohydride, sodium cyanoborohydride, lithium aluminium hydride. A most specific reducing agent is sodium borohydride.

In one embodiment, the second and third solvents used in steps-(b) and (c) are, each independently, selected from the group consisting of n-pentane, n-hexane, n-heptane, cyclohexane, toluene, xylene, dichloromethane, ethylene dichloride, and mixtures thereof; and more specifically selected from the group consisting of n-pentane, n-hexane, n-heptane, cyclohexane, toluene, xylene, and mixtures thereof.

In another embodiment, the reducing agent in step-(b) is used in an amount of about 1 to 4 equivalents, specifically about 3 to 4 equivalents, with respect to the (−)-(S)-5-methoxy-2-[N-2-(2-thienyl)ethylamino]tetralin of formula III in order to ensure a proper course of the reaction.

In another embodiment, the propionic acid or propionyl halide in step-(b) is used in an amount of about 2 to 8 equivalents, specifically about 6.5 to 7.5 equivalents, with respect to the (−)-(S)-5-methoxy-2-[N-2-(2-thienyl)ethylamino]tetralin of formula III in order to ensure a proper course of the reaction.

In one embodiment, the compound of formula III is used in the form of an acid addition salt or free base, and specifically in the form of free base.

In another embodiment, the reaction in step-(b) is carried out at a temperature of about 0° C. to the reflux temperature of the solvent used, specifically at a temperature of about 50° C. to about 110° C., and more specifically at a temperature of about 80° C. to about 100° C. The reaction is carried out for at least 1 hour, specifically for about 2 hours to about 8 hours, and more specifically for about 3 hours to about 5 hours. The reaction mass obtained after completion of the reaction may be quenched with water.

The reaction mass containing the (−)-(S)-5-methoxy-2-[N-n-propyl-N-2-(2-thienyl)ethylamino]tetralin of formula II obtained in step-(b) may be subjected to usual work up methods as described above, and then converted into its acid addition salt by reacting with a an acid in a suitable solvent.

The acid addition salts of the compound of formula II is derived from a therapeutically acceptable acid selected from the group as described above. A specific acid addition salt of the compound of formula II is hydrochloride salt.

In another embodiment, the (−)-(S)-5-methoxy-2-[N-n-propyl-N-2-(2-thienyl)ethylamino]tetralin of formula II formed in step-(b) is isolated as a solid, preferably in the form of its hydrochloride salt, from a suitable organic solvent by the methods as described above.

The organic solvent used for isolating the compound of formula II is selected from the group as described above.

In yet another embodiment, the reaction mass containing the (−)-(S)-5-methoxy-2-[N-n-propyl-N-2-(2-thienyl)ethylamino]tetralin of formula II obtained after completion of the reaction is quenched with water at a temperature of about 5° C. to about 30° C., specifically at a temperature of about 10° C. to 15° C. Aqueous sodium hydroxide solution is added to the reaction mixture to adjust the pH between 7.5 and 12, specifically 7.5 to 8.0, at a temperature of about 10° C. to about 15° C. The resulting organic layer is separated and the aqueous layer is extracted with a hydrocarbon solvent, preferably toluene. The combined organic layer is optionally washed with water followed by distillation to yield an oily residue. Concentrated hydrochloric acid and ethyl acetate are added to the oily residue and the pH of the reaction mixture is adjusted to between 1 and 2 using concentrated hydrochloric acid. The acidified mixture is optionally heated at a temperature of about 45° C. to about 110° C., specifically at about 70° C. to 80° C., for 10 minutes to 15 minutes prior to the vacuum distillation to obtain a residue. The resulting residue is dissolved in a polar organic solvent, preferably ethyl acetate, and heated to reflux for about 1 hour to about 3 hours, specifically for 1 hour. Finally, the solution is allowed to cool at a temperature of about 0° C. to about 35° C., specifically at about 0° C. to 5° C., to form a precipitate. The product is collected by the filtration. The wet cake is optionally washed with ethyl acetate preferably with chilled ethyl acetate. The obtained product is dried in the air oven at 50° C. to 55° C. for about 4 hours to about 8 hours, and more preferably for 4 hours.

In one embodiment, the Lewis acid used in step-(c) is selected from the group consisting of aluminium chloride, calcium chloride and zinc chloride, and a most specific Lewis acid is aluminium chloride.

In another embodiment, the Lewis acid in step-(c) is used in an amount of about 1 to 5 equivalents, specifically about 3 to 4 equivalents, with respect to the (−)-(S)-5-methoxy-2-[N-n-propyl-N-2-(2-thienyl)ethylamino]tetralin of formula II in order to ensure a proper course of the reaction.

In another embodiment, the thiourea in step-(c) is used in an amount of about 1 to 4 equivalents, specifically about 2.5 to 3.5 equivalents, with respect to the (−)-(S)-5-methoxy-2-[N-n-propyl-N-2-(2-thienyl)ethylamino]tetralin of formula II in order to ensure a proper course of the reaction.

In one embodiment, the compound of formula II is used in the form of an acid addition salt. Specific acid addition salts of the compound of formula II are hydrochloride, hydrobromide, and most specifically hydrochloride salt.

In another embodiment, the reaction in step-(c) is carried out at a temperature of about 0° C. to the reflux temperature of the solvent used, specifically at a temperature of about 45° C. to about 100° C., and more specifically at a temperature of about 55° C. to about 65° C. The reaction is specifically carried out for at least 1 hour, more specifically for about 4 hours to about 14 hours, and most specifically for about 9 hours to about 12 hours.

In one embodiment, the reaction mass obtained after completion of the reaction in step-(c) is cooled at a temperature of below about 40° C., specifically at about 10° C. to about 20° C., and the cooled reaction mass is quenched with a mixture of water and an aqueous ammonia or a mixture of an alcohol solvent and aqueous ammonia.

In another embodiment, the reaction mass containing the rotigotine of formula I obtained in step-(c) may be subjected to usual work up methods as described above, and then converted into its pharmaceutically acceptable acid addition salt by reacting with an acid in a solvent selected from the group comprising water, a alcohol, a chlorinated hydrocarbon, a hydrocarbon, a ketone, a nitrile, an ester, an ether, a polar aprotic solvents, and mixtures thereof.

The pharmaceutically acceptable acid addition salts of rotigotine of formula I are derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, and tartaric acid. A specific pharmaceutically acceptable acid addition salt of rotigotine is hydrochloride.

In yet another embodiment, the rotigotine of formula I formed in step-(c) is isolated as a solid in the form of its hydrochloride salt from a solvent by methods such as such as cooling, seeding, partial removal of the solvent from the solution, by adding an anti-solvent to the solution, evaporation, vacuum drying, spray drying, freeze drying, or a combination thereof.

Specifically, the solvent used to isolate the rotigotine of formula I or a pharmaceutically acceptable acid addition salt thereof is selected from the group consisting of water, acetone, methanol, ethanol, n-propanol, isopropanol, ethyl acetate, dichloromethane, n-pentane, n-hexane, n-heptane, cyclohexane, toluene, and mixture thereof.

In still another embodiment, the reaction mass containing the rotigotine of formula I obtained after completion of the reaction in step-(c) is cooled at a temperature of about 0° C. to about 40° C., specifically at about 10° C. to 20° C. The cooled reaction mixture is quenched with a mixture of water and aqueous ammonia or a mixture of alcohol solvent and aqueous ammonia, to adjust the pH of the reaction mixture to 8 to 9. A specific alcohol solvent is methanol. The resulting sludge is then filtered and optionally washed with a suitable organic solvent, preferably toluene and finally with water. The resulting filtrate is separated and the separated organic layer is washed with water. The organic solvent is distilled under vacuum at a temperature of about 55° C. to 60° C. and the resulting oil is dissolved in an organic solvent selected from the group consisting of a hydrocarbon, a haloalkane solvent and mixtures thereof; specifically toluene, dichloromethane, and mixtures thereof; and more specifically dichloromethane. Dry hydrochloric acid gas is bubbled through the obtained solution to reduce the pH to 1 to 2. The acidified solution is then heated to reflux for at least 30 minutes, and cooled to a temperature of about 10° C. to about 30° C., specifically at a temperature of about 15° C. to 20° C. The obtained product is collected by filtration and the resulting wet cake is optionally washed with dichloromethane.

In one embodiment, the purification of the rotigotine pharmaceutically acceptable acid addition salt in step-(d) is carried out by the methods disclosed hereinafter.

Exemplary fourth solvents used in step-(d) include, but are not limited to, water, an amide solvent, an alcohol, a ketone, a nitrile, and mixtures thereof. Specifically, the fourth solvent is selected from the group consisting of water, N,N-dimethylacetamide, N,N-dimethylformamide, acetone, methanol, ethanol, isopropanol, and mixtures thereof; and most specifically a mixture of N,N-dimethylacetamide and isopropanol.

Exemplary pharmaceutically acceptable salts of rotigotine include, but are not limited to, hydrochloride, hydrobromide, oxalate, maleate, fumarate, mesylate, besylate, tosylate, tartrate; and a specific pharmaceutically acceptable salt is rotigotine hydrochloride.

Rotigotine free base can be prepared in high purity by using the highly pure rotigotine pharmaceutically acceptable acid addition salt obtained by the methods disclosed herein, by known methods or by the method disclosed hereinafter.

The highly pure rotigotine or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that provides the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

The total purity of the rotigotine or a pharmaceutically acceptable salt thereof, preferably rotigotine hydrochloride, obtained by the process disclosed herein is of greater than about 99%, specifically greater than about 99.5%, more specifically greater than about 99.9%, and most specifically greater than about 99.95% as measured by HPLC. For example, the total purity of the rotigotine or a pharmaceutically acceptable salt thereof of the present invention can be about 99% to about 99.95%, or about 99.5% to about 99.99%.

2-(2-Thienyl)-ethyl para-toluenesulfonate of formula V used as starting material in step-(a) may be obtained by processes described in the prior art, for example by the process described in the U.S. Pat. No. 4,127,580 or by the process exemplified herein.

(S)-2-Amino-5-methoxytetraline of formula IV used as starting material in step-(a) may be obtained by processes described in the prior art, for example by the process described in the Organic Process Research & Development 2005, 9, 30-38.

According to another aspect, there is provided a process for the preparation of (−)-(S)-5-hydroxy-2-[N-n-propyl-N-2-(2-thienyl)ethylamino]tetralin (Rotigotine) of formula I:

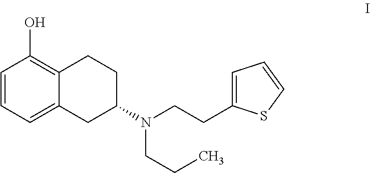

or a pharmaceutically acceptable salt thereof, comprising demethylating the compound of formula II:

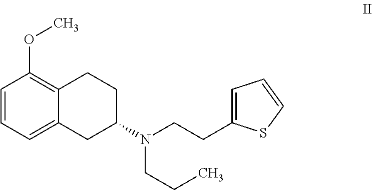

or an acid addition salt thereof, with a Lewis acid in the presence of thiourea in an organic solvent to produce rotigotine of formula I or a pharmaceutically acceptable salt thereof.

In one embodiment, the Lewis acid is selected from the group as described above. A most specific Lewis acid is aluminium chloride.

In another embodiment, the organic solvent is selected from the group consisting of a chlorinated hydrocarbon, a hydrocarbon, and mixtures thereof. Specifically, the organic solvent is selected from the group consisting of n-pentane, n-hexane, n-heptane, cyclohexane, toluene, xylene, dichloromethane, ethylene dichloride, and mixtures thereof; more specifically the organic solvent is selected from the group consisting of n-pentane, n-hexane, n-heptane, cyclohexane, toluene, xylene, and mixtures thereof; and a most specific organic solvent is toluene.

In one embodiment, the compound of formula II is used in the form of an acid addition salt. Specific acid addition salts of the compound of formula II are hydrochloride, hydrobromide, and most specifically the hydrochloride salt.

In another embodiment, the reaction is carried out at a temperature of about 0° C. to the reflux temperature of the solvent used, specifically at a temperature of about 45° C. to about 100° C., and more specifically at a temperature of about 55° C. to about 65° C. The reaction is carried out for at least 1 hour, preferably from about 4 hours to about 14 hours, and more preferably from about 9 hours to about 12 hours.

The reaction mass containing the rotigotine of formula I obtained may be subjected to usual work up, and then converted into its pharmaceutically acceptable acid addition salt by the methods as described above.

In one embodiment, the pharmaceutically acceptable salts of the rotigotine of formula I are derived from a therapeutically acceptable acid selected from the group as described above.

In another embodiment, the rotigotine of formula I or a pharmaceutically acceptable salt thereof, preferably the hydrochloride salt, is isolated as a solid and further dried by the methods described herein.

According to another aspect, there is provided a process for purifying rotigotine hydrochloride, comprising:
a) providing a solution of crude rotigotine hydrochloride in a first solvent or in a solvent medium comprising the first solvent and a second solvent, wherein the first solvent is selected from the group consisting of an amide, a ketone, a nitrile and mixtures thereof, and wherein the second solvent is selected from the group consisting, of water, an alcohol and mixtures thereof;
b) optionally, subjecting the solvent solution to carbon treatment or silica gel treatment; and
c) isolating the highly pure rotigotine hydrochloride from the solution and optionally converting the rotigotine hydrochloride obtained into highly pure rotigotine free base.

The term “highly pure rotigotine hydrochloride” refers to the rotigotine hydrochloride having a total purity of greater than about 99%, specifically greater than about 99.5%, more specifically greater than about 99.8%, and most specifically greater than about 99.9% (measured by HPLC).

The term ‘crude rotigotine hydrochloride’ in the specification refers to rotigotine hydrochloride having a total purity of less than about 99%, specifically less than about 98%, and most specifically less than about 97%, as measured by HPLC.

In one embodiment, the first solvent used in step-(a) is selected from the group consisting of N,N-dimethylacetamide, N,N-dimethylformamide, acetone, methyl ethyl ketone, methyl isobutyl ketone, methyl tert-butyl ketone, acetonitrile, propionitirle, and mixtures thereof; and most specifically, the first solvent is selected from the group consisting of N,N-dimethylacetamide, N,N-dimethylformamide, acetone, and mixtures thereof.

In another embodiment, the second solvent used in step-(a) is selected from the group consisting of water, methanol, ethanol, isopropanol, n-propanol, n-butanol, tert-butanol, amyl alcohol, hexanol, and mixtures thereof; and most specifically, the second solvent is selected from the group consisting of water, methanol, isopropanol, and mixtures thereof.

In yet another embodiment, the solvent medium used in step-(a) is aqueous acetone or a mixture of N,N-dimethylacetamide and isopropanol.

Step-(a) of providing a solution of crude rotigotine hydrochloride includes dissolving crude rotigotine hydrochloride in the solvent or the solvent medium, or obtaining an existing solution from a previous processing step.

In one embodiment, the crude rotigotine hydrochloride is dissolved in the solvent or the solvent medium at a temperature of above about 20° C., specifically at about 25° C. to about 100° C., and more specifically at about 50° C. to about 80° C.

The carbon treatment or silica gel treatment in step-(b) is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing rotigotine hydrochloride by removing charcoal or silica gel. Specifically, the finely powdered carbon is an active carbon. A specific mesh size of silica gel is 40-500 mesh, and more specifically 60-120 mesh.

In another embodiment, the solution obtained in step-(a) or step-(b) is stirred for at least 20 minutes, specifically for about 30 minutes to about 4 hours, at a temperature of about 20° C. to about 35° C.

The isolation of highly pure rotigotine hydrochloride in step-(c) is carried out, for example, by forcible or spontaneous crystallization.

Spontaneous crystallization refers to crystallization without the help of an external aid such as seeding, cooling etc., and forcible crystallization refers to crystallization with the help of an external aid.

Forcible crystallization is initiated by methods such as cooling, seeding, partial removal of the solvent from the solution, by adding an anti-solvent to the solution, or a combination thereof.

The term “anti-solvent” refers to a solvent which when added to an existing solution of a substance reduces the solubility of the substance.

Exemplary anti-solvents include, but are not limited to, an ether, a hydrocarbon, and mixtures thereof. Specifically, the anti-solvent is selected from the group consisting of diisopropyl ether, diethyl ether, tetrahydrofuran, dioxane, n-pentane, n-hexane, n-heptane and their isomers, cyclohexane, toluene, xylene, and mixtures thereof.

In one embodiment, the crystallization is carried out by cooling the solution while stirring at a temperature of below 20° C. for at least 15 minutes, specifically at about 0° C. to about 15° C. for about 30 minutes to about 20 hours, and more specifically at about 0° C. to about 10° C. for about 1 hour to about 10 hours.

The highly pure rotigotine hydrochloride obtained in step-(c) is recovered by techniques such as filtration, filtration under vacuum, decantation, centrifugation, or a combination thereof. In one embodiment, the rotigotine hydrochloride is recovered by filtration employing a filtration media of, for example, a silica gel or celite.

In another embodiment, the highly pure rotigotine hydrochloride obtained in step-(c) is further dried by the methods as described above.

According to another aspect, there is provided a highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities.

As used herein, “highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities” refers to rotigotine or a pharmaceutically acceptable salt thereof comprising one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities, each one, in an amount of less than about 0.1 area-% as measured by HPLC. Specifically, the rotigotine, as disclosed herein, contains less than about 0.08 area-%, more specifically less than about 0.05 area-%, still more specifically less than about 0.02 area-% of one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities, and most specifically is essentially free of one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities.

In one embodiment, the highly pure rotigotine or a pharmaceutically acceptable salt thereof disclosed herein comprises one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities each in an amount of about 0.01 area-% to about 0.1 area-%, specifically in an amount of about 0.01 area-% to about 0.05 area-%, as measured by HPLC.

The term “rotigotine or a pharmaceutically acceptable salt thereof essentially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities” refers to rotigotine or a pharmaceutically acceptable salt thereof contains a non-detectable amount of one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities as measured by HPLC.

According to another aspect, there is provided a process for the preparation of highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities, comprising:
a) providing a first solution of crude rotigotine free base in a first solvent, wherein the first solvent is a chlorinated hydrocarbon solvent or a solvent medium comprising a chlorinated hydrocarbon solvent and an ester solvent;
b) subjected the first solution to silica gel treatment to provide a second solution; and
c) substantially removing the solvent from the second solution to produce an oily mass containing rotigotine free base;
d) combining the oily mass obtained in step-(c) with a second solvent to produce a third solution, and
e) isolating the highly pure rotigotine free base substantially free of impurities from the third solution, and optionally converting the highly pure rotigotine free base obtained into a pharmaceutically acceptable salt thereof.

The term “crude rotigotine free base” as used herein refers to the rotigotine free base containing greater than about 0.1 area-%, more specifically greater than about 0.15 area-%, still more specifically greater than about 0.2 area-% and most specifically greater than about 0.3 area-% of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities.

In one embodiment, the first solvent used in step-(a) is selected from the group consisting of dichloromethane, ethylene dichloride, chloroform, carbon tetrachloride, ethyl acetate, methyl acetate, isopropyl acetate, tert-butyl methyl acetate, ethyl formate, and mixtures thereof. Specifically, the first solvent is dichloromethane or a mixture of dichloromethane and ethyl acetate.

Step-(a) of providing a solution of crude rotigotine free base includes dissolving or extracting the crude rotigotine free base in the first solvent, or obtaining an existing solution from a previous processing step.

In one embodiment, the crude rotigotine free base is dissolved in the first solvent at a temperature of above about 25° C., specifically at about 25° C. to about 110° C., and more specifically at about 40° C. to about 80° C.

Alternatively, the first solution in step-(a) is prepared by treating an acid addition salt of rotigotine with a base to produce rotigotine free base followed by extracting or dissolving the rotigotine free base in the first solvent at a temperature of above about 25° C., specifically at about 25° C. to about 110° C., and more specifically at about 40° C. to about 80° C.

In another embodiment, the acid addition salt of rotigotine is derived from a therapeutically acceptable acid selected from the group as described above. A specific acid addition salt of rotigotine is the hydrochloride salt.

The treatment of an acid addition salt with a base is carried out in a solvent and the selection of solvent is not critical. A wide variety of solvents such as water, a chlorinated hydrocarbon, an alcohol, a ketone, a hydrocarbon, an ester, an ether solvent, and mixtures thereof, can be used.

In one embodiment, the base is an organic or inorganic base selected from the group as described above. A most specific base is aqueous ammonia.

The first solution obtained in step-(a) is optionally stirred at a temperature of about 25° C. to about 110° C. for at least 15 minutes and specifically at a temperature of about 40° C. to about 80° C. for about 20 minutes to about 8 hours.

In one embodiment, the silica gel treatment is carried out by methods such as stirring the solution with silica gel, by column chromatography, passing the solution through silica bed, or by circulation through silica cartridge repetitively.

In another embodiment, the silica gel treatment in step-(b) is carried out by stirring the solution with silica gel at a temperature of below about 70° C. for at least 15 minutes, specifically at a temperature of about 40° C. to about 70° C. for at least 30 minutes; and filtering the resulting mixture through a filtration bed to obtain the second solution containing rotigotine free base by removing silica gel. A specific mesh size of silica gel is 35-500 mesh, and more specifically 60-120 mesh.

The term “substantially removing” the solvent refers to at least 60%, specifically greater than about 85%, more specifically greater than about 90%, still more specifically greater than about 99%, and most specifically essentially complete (100%), removal of the solvent from the solvent solution.

Removal of solvent in step-(c) is accomplished, for example, by substantially complete evaporation of the solvent, concentrating the solution or distillation of solvent, under inert atmosphere.

In one embodiment, the solvent is removed by evaporation. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

Exemplary second solvents used in step-(d) include, but are not limited to, an ether, an aliphatic or alicyclic hydrocarbon solvent, and mixtures thereof. Specifically, the second solvent is selected from the group consisting of diisopropyl ether, diethyl ether, tetrahydrofuran, dioxane, n-pentane, n-hexane, n-heptane and their isomers, cyclohexane, and mixtures thereof; and most specifically, the second solvent is n-heptane.

Combining of the oily mass with the second solvent in step-(d) is done in a suitable order, for example, the oily mass is added to the second solvent, or alternatively, the second solvent is added to the oily mass. The addition is, for example, carried out drop wise or in one portion or in more than one portion. The addition is specifically carried out at a temperature of above about 25° C., more specifically at about 30° C. to about 110° C., and most specifically at about 40° C. to about 80° C. under stirring. After completion of the addition process, the resulting mass is heated and stirred at a temperature of above about 50° C. for at least 10 minutes, specifically at about 55° C. to about 100° C. for about 20 minutes to about 10 hours, and more specifically at a temperature of about 60° C. to about 80° C. for about 30 minutes to about 4 hours, to produce the third solution.

The isolation of highly pure rotigotine free substantially free of impurities in step-(e) is carried out by forcible or spontaneous crystallization methods as described above.

In one embodiment, the crystallization is carried out by cooling the solution while stirring at a temperature of below 30° C. for at least 15 minutes, specifically at about 0° C. to about 25° C. for about 30 minutes to about 20 hours, and more specifically at about 0° C. to about 10° C. for about 1 hour to about 10 hours.

The highly pure rotigotine free base substantially free of impurities obtained in step-(e) is recovered by techniques such as filtration, filtration under vacuum, decantation, centrifugation, or a combination thereof. In one embodiment, the rotigotine free base is recovered by filtration employing a filtration media of, for example, a silica gel or celite. In another embodiment, the rotigotine free base obtained is further dried by the methods described above.

Pharmaceutically acceptable salts of rotigotine can be prepared in high purity by using the highly pure rotigotine free base substantially free of impurities obtained by the method disclosed herein, by known methods.

Further encompassed herein is the use of the highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities for the manufacture of a pharmaceutical composition together with a pharmaceutically acceptable carrier.

A specific pharmaceutical composition of highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities is selected from a solid dosage form and an oral suspension. In one embodiment, the highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities has a D₉₀ particle size of less than or equal to about 400 microns, specifically less than or equal to about 300 microns, more specifically less than or equal to about 100 microns, still more specifically less than or equal to about 60 microns, and most specifically less than or equal to about 15 microns.

In another embodiment, the particle sizes of the highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from disorders of the central nervous, cardiovascular and endocrine systems such as Parkinson's disease and related disorders, hypertension and hyper-prolactinemia, comprising administering a therapeutically effective amount of the highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities, or a pharmaceutical composition that comprises a therapeutically effective amount of highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities, along with pharmaceutically acceptable excipients.

According to another aspect, there is provided pharmaceutical compositions comprising highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities prepared according to the processes disclosed herein and one or more pharmaceutically acceptable excipients.

According to another aspect, there is provided a process for preparing a pharmaceutical formulation comprising combining highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities prepared according to processes disclosed herein, with one or more pharmaceutically acceptable excipients.

Yet in another embodiment, pharmaceutical compositions comprise at least a therapeutically effective amount of highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure rotigotine or a pharmaceutically acceptable salt thereof substantially free of at least one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### EXPERIMENTAL

### HPLC Method for Measuring Chemical Purity:

### Chromatographic Parameters:

Column: Unison (250×4.6 mm 5 μ) Make: Imtakt Corporation, Part No.
   US006 or its equivalent.
Detector: UV at 233 nm
Flow rate: 1.0 mL/min
Injection volume: 10.0 μL
Run time: 65 min
Oven temperature: 35° C.
Diluent: Buffer:Acetonitrile (70:30 v/v).

### Buffer Preparation:

About 2.72 g of potassium dihydrogen phosphate was weighed and added to 1000 mL of water and adjusted the pH to 2.30±0.05 with dilute orthophosphoric acid and followed by filtration through 0.45 μm or finer porosity membrane and degassing.
Mobile Phase-A: Buffer
Mobile Phase-B: Acetonitrile:Methanol (60:40 v/v).

The following examples are given for the purpose of illustrating the present disclosure and should not be considered as limitation on the scope or spirit of the disclosure.

### EXAMPLES

### Example 1

### Preparation of 2-(2-Thienyl)-ethyl para-toluenesulfonate

A mixture of p-toluenesulfonyl chloride (328 gm) and 2-(2-thienyl)-ethanol (200 gm) in methyl ethyl ketone (1000 ml) was cooled to 0° C. This was followed by drop wise addition of triethyl amine (283.1 ml) at 0-5° C. over a period of 1 to 2 hours, and the reaction mass was stirred for 12 to 15 hours at 25-30° C. The resulting mass was filtered and washed with methyl ethyl ketone (500 ml). The resulting organic layer was washed with water (500 ml) followed by washings with saturated sodium bicarbonate solution (500 ml) and brine solution (500 ml). The resulting organic layer was distilled under vacuum at below 50° C. to give 2-(2-thienyl)-ethyl para-toluenesulfonate as an oily mass (Weight of the oil: 505 gm; Purity by HPLC: 97%).

### Example 2

### Preparation of (−)-(S)-5-methoxy-2-[N-2-(2-thienyl)ethylamino]tetralin hydrochloride

(S)-2-Amino-5-methoxytetraline hydrochloride (100 gm), 2-(2-thienyl)-ethyl para-toluenesulfonate (158.52 gm) and potassium carbonate (142.25 g) were added to acetonitrile (1000 ml) under stirring at 20-25° C., and the resulting mixture was heated at 80-85° C. for 20 to 22 hours. The resulting mass was filtered at 60-65° C., washed with acetonitrile (260 ml) and the resulting filtrate was distilled under vacuum at 50-55° C. to remove acetonitrile. Water (400 ml) was added to the resulting residue, followed by the addition of ethyl acetate (400 ml) and then stirred for 30 minutes. The layers were separated and the aqueous layer was extracted with ethyl acetate (200 ml). The organic layers were combined, and the total organic layer was washed with water (200 ml). Concentrated hydrochloric acid (40 ml) was added to the resulting organic layer and then heated to 80-85° C. for 15 to 20 min. The resulting mass was cooled to 0-5° C. and then stirred for 2 hours at the same temperature. The resulting solid was filtered, washed with pre-cooled ethyl acetate (150 ml) and then dried the material at 50-55° C. for 3-4 hours to give 107 gm of (−)-(S)-5-methoxy-2-[N-2-(2-thienyl)ethylamino]tetralin hydrochloride [Yield 70%; Purity by HPLC: 99.96%; SOR=−55.7 (C=1, methanol)].

### Example 3

### Preparation of (−)-(S)-5-methoxy-2-[N-n-propyl-N-2-(2-thienyl)ethylamino]tetralin hydrochloride

Propionic acid (180.4 gm) was added to toluene (800 ml) and the resulting mixture was cooled to 0° C. Sodium borohydride (46 gm) was added to the reaction mixture at 0-5° C. and stirred for 30 minutes at 0-5° C., followed by stirring for 1 hour at 20-25° C. The reaction mixture was followed by the addition of a solution of (−)-(S)-5-methoxy-2-[N-2-(2-thienyl)ethyl amino]tetralin (100 gm) in toluene (200 ml) at 20-25° C. The resulting mixture was heated at 90-95° C. for 3 hours. The resulting mass was cooled to 0-5° C., followed by the addition of ice cooled water (500 ml) at 0-5° C. and 10% NaOH solution (375 ml, to adjust pH 7.5 to 8.0). The resulting mixture was stirred for 20 to 30 minutes at 20-25° C., followed by separation of the layers. The aqueous layer was extracted with toluene (200 ml) and the resulting total organic layer was washed with water (500 ml) and followed by distillation of toluene under vacuum at 50-55° C. Ethyl acetate (200 ml) and hydrochloric acid (40 ml) were added to the residue followed by distillation under vacuum. Ethyl acetate (500 ml) was added to the resulting residue, the resulting mixture was heated to reflux and then stirred for 30 minutes. The resulting mass was cooled to 0° C. and stirred for 2 to 3 hours at the same temperature. The separated solid was filtered, washed with pre cooled ethyl acetate (150 ml), and dried at 50 to 55° C. in air oven to give 108 gm of (−)-(S)-5-methoxy-2-[N-n-propyl-N-2-(2-thienyl)ethylamino]tetralin hydrochloride (Yield 84.8%; Purity by HPLC: 99.87%).

### Example 4

### Preparation of (−)-(S)-5-Hydroxy-2-[N-n-propyl-N-2-(2-thienyl)ethylamino]tetralin hydrochloride (Rotigotine hydrochloride)

Aluminium chloride (14.5 gm) and thiourea (6.2 gm) were added to toluene (110 ml) and the mixture was stirred for 30 minutes at 20-25° C. The mixture was followed by the addition of (−)-(S)-5-methoxy-2-[N-n-propyl-N-2-(2-thienyl)ethylamino]tetralin hydrochloride (10 gm) and stirred at 60-65° C. till completion of the reaction. The resulting mass was cooled to 15-20° C., followed by the addition of a mixture of water (140 ml) and ammonia solution (70 ml) at below 30° C. to adjust the pH to 8 to 9. The reaction mixture was filtered through celite and washed the celite with toluene (50 ml) followed by separation of the layers. The toluene layer was washed with water (2×50 ml) and dried over potassium carbonate, followed by distillation of toluene under vacuum at 50-55° C. Dichloromethane (50 ml) was added to the resulting residue and then hydrogen chloride gas was bubbled into the reaction mass until saturation and then stirred for 1 hour. The separated solid was filtered at 0-5° C., washed with dichloromethane (15 ml) and then dried at 50-55° C. to give 7.2 gm of rotigotine hydrochloride (Yield: 82%; Purity by HPLC: 96.5%).

### Example 5

### Purification of (−)-(S)-5-Hydroxy-2-[N-n-propyl-N-2-(2-thienyl)ethylamino]tetralin hydrochloride (Rotigotine hydrochloride)

Rotigotine hydrochloride (1 gm, obtained in example 4) was added to 10% aqueous acetone (6 ml) and the mixture was heated at 65-70° C. followed by the addition of 10% aqueous acetone (8 ml) to get clear solution. The solution was gradually cooled to 20-25° C. and stirred for 15 hours. The reaction mixture was further cooled to 10-15° C., the separated solid was filtered, and then dried at 55° C. in air oven to give 0.4 gm of pure rotigotine hydrochloride (Yield: 40%; Purity by HPLC: 99.47%).

### Example 6

### Purification of (−)-(S)-5-Hydroxy-2-[N-n-propyl-N-2-(2-thienyl)ethylamino]tetralin hydrochloride (Rotigotine hydrochloride)

A mixture of rotigotine hydrochloride (5 gm, obtained in example 4) and dimethylacetamide (10 ml) was heated at 80-85° C. to get clear solution. The resulting mass was followed by the addition of isopropyl alcohol (20 ml) to get clear solution. The resulting clear solution was gradually cooled to 20-25° C. and stirred for 15-20 hours. The reaction mixture was further cooled to 5-10° C., the separated solid was filtered and washed with pre-cooled isopropanol and then dried at 55° C. in air oven to give 3.5 gm of pure rotigotine hydrochloride (Yield: 70%; Purity by HPLC: 99.85%).

### Example 7

### Preparation of (−)-(S)-5-Hydroxy-2-[N-n-propyl-N-2-(2-thienyl)ethylamino]tetralin (Rotigotine)

Pure rotigotine hydrochloride (5 gm, obtained in example 6) was added to water (25 ml) and the pH of the resulting mixture was adjusted to 8 to 10 using ammonia solution. The resulting mass was extracted with dichloromethane (25 ml) and then distilled with dichloromethane to dryness followed by co-distillation with n-heptane (10 ml). Heptane (30 ml) was added to the resulting residue and heated at 60 to 70° C. The resulting solution was cooled to 0-5° C. and then stirred for 2 hours. The separated solid was filtered, washed with heptane (5 ml) and then dried under vacuum at 40° C. for 4 hours to give 3.8 gm of rotigotine (Yield 85%; Purity by HPLC: 99.91%; Chiral purity: 100%).

### Example 8

### Preparation of (−)-(S)-5-Hydroxy-2-[N-n-propyl-N-2-(2-thienyl)ethylamino]tetralin hydrochloride (Rotigotine hydrochloride)

Step-I:

Propionic acid (4.5 gm) was added to toluene (75 ml) at 20 to 25° C. followed by the addition of sodium borohydride (2.4 gm) at 20 to 25° C. and stirred for 30 minutes. A solution of (−)-(S)-5-Methoxy-2-[N-2-(2-thienyl)ethylamino]tetraline (5 gm) in toluene (10 ml) was added to the above reaction mass at 20-25° C. The reaction mixture was heated at 100-105° C. for 3 hours and the resulting mass was cooled to 20-25° C. This was followed by addition of 50% aqueous sodium hydroxide (20 ml) and stirring for 20-30 minutes followed by the separation of layers. The aqueous layer was extracted with toluene (25 ml). The organic layers were combined and washed with water (25 ml) followed by distillation of toluene under vacuum to give (−)-(S)-5-methoxy-2-[N-n-propyl-N-2-(2-thienyl) ethylamino]tetralin.

Step-II:

Aluminium chloride (7.6 gm) and thiourea (3.4 gm) were added to toluene (50 ml) and stirred for 30 minutes at 20-25° C. The mixture was followed by the addition of a solution of (−)-(S)-5-methoxy-2-[N-n-propyl-N-2-(2-thienyl)ethylamino]tetralin (5 gm, obtained in step-I) in toluene (10 ml) and heated at 100-105° C. for 3 hours. The resulting mass was cooled to 20-25° C. followed by drop wise addition of ammonia solution (10 ml) to adjust the pH to 8 to 9. Water (30 ml) was added to the resulting mixture, and then the mixture was filtered over a celite bed. The layers were separated and the toluene layer was washed with water (25 ml) and then dried over potassium carbonate. Hydrogen chloride gas was bubbled into the resulting mass, heated to reflux and then stirred for 1 hour. The resulting mass was cooled to 20 to 25° C. and stirred for 2 hours. The separated solid was filtered, washed with toluene (10 ml) and then dried at 50-55° C. to give 2.5 gm of rotigotine hydrochloride

### Example 9

### Preparation of (−)-(S)-5-Hydroxy-2-[N-n-propyl-N-2-(2-thienyl)ethylamino]tetralin (Rotigotine)

Aluminium chloride (5.32 gm) and thiourea (2.3 gm) were added to toluene (40 ml) and then stirred for 30 minutes at 20 to 25° C. The mixture was followed by the addition of (−)-(S)-5-methoxy-2-[N-n-propyl-N-2-(2-thienyl)ethylamino]tetralin hydrochloride (4 gm) and then refluxed for 2 hours. The resulting mass was cooled to 10-15° C. followed by drop wise addition of water (15 ml) and ammonia solution (12 ml). The resulting mixture was stirred for 1 hour at 20 to 25° C., followed by the addition of water (12 ml) and toluene (25 ml). The resulting mixture was filtered, washed the wet cake with water (10 ml) and toluene (10 ml). The resulting layers were separated, washed the toluene layer with water (25 ml) and then dried over potassium carbonate. Toluene was distilled off under vacuum at 50-55° C. followed by co-distillation with n-heptane (30 ml). Heptane (28 ml) was added to the residue and stirred for 1 hour. The separated solid was filtered, washed with heptane (5 ml) and then dried at 40° C. to give 3 gm of rotigotine (Yield: 86%).

### Example 10

### Preparation of Pure Rotigotine Free Base

Water (5 ml) and aqueous ammonia solution (2 ml) were added to rotigotine hydrochloride (1 gm) and the resulting mass was extracted two times with dichloromethane (5 ml×2). The combined organic layer was washed with water (10 ml) and then dried over sodium sulphate. The organic layer was passed through a silica bed (20 gm) and the silica bed was washed with dichloromethane (600 ml) and followed by distillation of dichloromethane to produce an oily mass. Heptane (15 ml) was added to the resulting oily mass and then heated at 60 to 70° C. to form a clear solution. The resulting solution was cooled to 25-30° C. and then stirred for 3 to 4 hours. The separated solid was filtered, washed with heptane and then dried in air oven at 50 to 55° C. to give 0.7 gm of rotigotine free base.

Content of Impurities:

Before Purification: HPLC Purity: 99.65%; ‘0.92 RRt’ impurity: 0.11%; and ‘0.79 RRt’ impurity: 0.07%;

After Purification: HPLC Purity: 99.81%; ‘0.92 RRt’ impurity: 0.03% and ‘0.79 RRt’ impurity: Below detection limit.

### Example 11

### Preparation of Pure Rotigotine Free Base

Water (5 ml) and aqueous ammonia solution (2 ml) were added to rotigotine hydrochloride (2 gm) and the resulting mass was extracted two times with dichloromethane (10 ml×2). The combined organic layer was washed with water (10 ml) and then dried over sodium sulphate. The organic layer was passed through a silica bed (40 gm) and the silica bed was washed with a mixture of dichloromethane and ethyl acetate (500 ml, 50:50), followed by distillation of the organic solvent to produce an oily mass. Heptane (20 ml) was added to the resulting oily mass and then heated at 60 to 70° C. to form a clear solution. The resulting solution was cooled to 25-30° C. and then stirred for 3 to 4 hours. The separated solid was filtered, washed with heptane and then dried in an air oven at 50 to 55° C. to give 1.6 gm of rotigotine free base.

Content of Impurities:

Before Purification: HPLC purity: 99.65%; ‘0.92 RRt’ impurity: 0.11%; and ‘0.79 RRt’ impurity: 0.07%.

After Purification: HPLC purity: 99.88%; ‘0.92 RRt’ impurity: 0.06% and ‘0.79 RRt’ impurity: 0.01%.

### Example 12

### Preparation of Pure Rotigotine Free Base

Water (5 ml) and aqueous ammonia solution (2 ml) were added to rotigotine hydrochloride (1 gm) and the resulting mass was extracted two times with dichloromethane (25 ml×2). The combined organic layer was washed with water (10 ml) and then dried over sodium sulphate. The organic layer was stirred with silica (2 gm) for 2 hours, and then the silica was filtered, followed by distillation of dichloromethane to produce an oily mass. Heptane (50 ml) was added to the resulting oily mass and then heated at 60 to 70° C. to form a clear solution. The resulting solution was cooled to 0-5° C. and then stirred for 2 hours. The separated solid was filtered, washed with heptane and then dried in an air oven at 50 to 55° C. to give 0.6 gm of rotigotine free base.

Content of Impurities:

Before Purification: HPLC purity: 99.65%; ‘0.92 RRt’ impurity: 0.11% and ‘0.79 RRt’ impurity: 0.07%.

After Purification: HPLC Purity: 99.77%; ‘0.92 RRt impurity: 0.06%; and ‘0.78 RRt’ impurity: 0.02%.

### Example 13

### Preparation of Pure Rotigotine Free Base

Water (400 ml) and aqueous ammonia solution (60 ml) were added to rotigotine hydrochloride (50 gm) and the resulting mass was extracted three times with dichloromethane (200 ml×2, 100 ml×1). The combined organic layer was washed with water (200 ml×2) and then dried over sodium sulphate. The organic layer was passed through a silica column (200 gm) and then washed the silica with dichloromethane (12 L), followed by distillation of dichloromethane to produce an oily mass. Heptane (400 ml) was added to the resulting oily mass and then heated at 60 to 70° C. to form a clear solution. The resulting solution was cooled to 25-30° C. and then stirred for 3 to 4 hours. The separated solid was filtered, washed with heptane and then dried in an air oven at 50 to 55° C. to give 41.5 gm of rotigotine free base.

Content of Impurities:

Before Purification: HPLC purity: 99.84%; ‘0.92 RRt’ impurity: 0.09%; and ‘0.79 RRt’ impurity: 0.03%.

After Purification: HPLC purity: 99.89%; ‘0.92 RRt’ impurity: 0.05%; and ‘0.79 RRt’ impurity: Below detection limit.

### Example 14

### Preparation of Pure Rotigotine Free Base

Water (465 ml) and aqueous ammonia solution (140 ml) were added to rotigotine hydrochloride (140 gm) and the resulting mass was extracted two times with dichloromethane (420 ml×2). The combined organic layer was washed with water (465 ml×2) and then dried over sodium sulphate. The organic layer was passed through silica column (650 gm) and then washed the silica with dichloromethane (2 L) followed by a mixture of dichloromethane and ethyl acetate (13 L, 70:30) and followed by distillation of the organic solvent to produce an oily mass. Heptane (1080 ml) was added to the resulting oily mass and then heated at 60 to 70° C. to form a clear solution. The resulting solution was cooled to 25-30° C. and then stirred for 3 to 4 hours. The separated solid was filtered, washed with heptane and then dried in air oven at 50 to 55° C. to give 119 gm of rotigotine free base.

Content of Impurities:

Before Purification: HPLC purity: 99.50%; ‘0.92 RRt impurity’: 0.05%; ‘0.79 RRt’ impurity: 0.03%; ‘1.12RRt’ impurity: 0.08%; ‘1.51 RRt’ impurity: 0.06%; ‘1.59 RRt’ impurity: 0.08%; ‘1.64 RRt’ impurity: 0.07%; and ‘1.79 RRt’ impurity: 0.06%.

After Purification: HPLC purity=99.88%; ‘0.92 RRt’ impurity: 0.05%.

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F 127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

As used herein, the term, “detectable” refers to a measurable quantity measured using an HPLC method having a detection limit of 0.01 area-%.

As used herein, in connection with amount of impurities in rotigotine or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for the preparation of (−)-(S)-5-hydroxy-2-[N-n-propyl-N-2-(2-thienyl)ethylamino]tetralin (Rotigotine) of formula I: 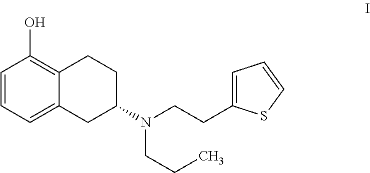
or a pharmaceutically acceptable acid addition salt thereof, comprising:
a) reacting (S)-2-amino-5-methoxytetraline of formula IV: 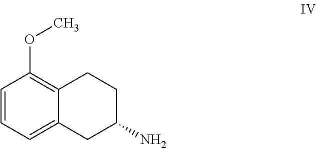
or an acid addition salt thereof with 2-(2-thienyl)-ethyl para-toluenesulfonate of formula V: 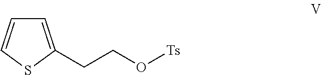
in the presence of a base in a first solvent to produce (−)-(S)-5-methoxy-2-[N-2-(2-thienyl)ethylamino]tetralin of formula III: 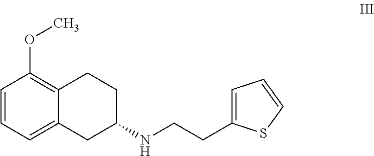
or an acid addition salt thereof;
b) reacting the compound of formula III or an acid addition salt thereof with propionic acid or propionyl halide in the presence of a reducing agent in a second solvent to produce (−)-(S)-5-methoxy-2-[N-n-propyl-N-2-(2-thienyl)ethylamino]tetralin of formula II: 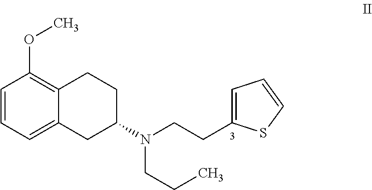
or an acid addition salt thereof;
c) demethylating the compound of formula II with a Lewis acid in the presence of thiourea in a third solvent to produce rotigotine of formula I or a pharmaceutically acceptable acid addition salt thereof; and
d) optionally, purifying the rotigotine pharmaceutically acceptable acid addition salt obtained in step-(c) with a fourth solvent to produce highly pure rotigotine pharmaceutically acceptable acid addition salt and then converting the purified rotigotine pharmaceutically acceptable acid addition salt into highly pure rotigotine free base.

2. The process of claim 1, wherein the first solvent used in step-(a) is selected from the group consisting of water, methanol, ethanol, n-propanol, isopropanol, n-butanol, isobutanol, tert-butanol, amyl alcohol, hexanol, acetonitrile, ethyl acetate, methyl acetate, isopropyl acetate, tert-butyl methyl acetate, ethyl formate, acetonitrile, dichloromethane, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; wherein the second and third solvents used in steps-(b) and (c) are, each independently, selected from the group consisting of n-pentane, n-hexane, n-heptane, cyclohexane, toluene, xylene, dichloromethane, ethylene dichloride, and mixtures thereof; and wherein the fourth solvents used in step-(d) is selected from the group consisting of water, an amide solvent, an alcohol, a ketone, a nitrile, and mixtures thereof.

3. The process of claim 2, wherein the first solvent is selected from the group consisting of acetonitrile, dimethyformamide, dimethylsulfoxide, tetrahydrofuran, toluene, dichloromethane, acetone, and mixtures thereof; wherein the second and third solvents are, each independently, selected from the group consisting of n-pentane, n-hexane, n-heptane, cyclohexane, toluene, xylene, and mixtures thereof; and wherein the fourth solvent is a mixture of N,N-dimethylacetamide and isopropanol.

4. The process of claim 1, wherein the base used in step-(a) is an organic or inorganic base selected from the group consisting of triethylamine, tributylamine, diisopropylethylamine, diethylamine, tert-butylamine, N-methylmorpholine, pyridine, 4-(N,N-dimethylamino)pyridine, ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide, potassium tert-butoxide, and mixtures thereof; wherein the acid addition salts of the compounds of formulae III and II are derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, and tartaric acid; wherein the propionyl halide used in step-(b) is propionyl chloride or propionyl bromide; wherein the reducing agent used in step-(b) is a metal hydride selected from the group consisting of sodium borohydride, sodium cyanoborohydride and lithium aluminium hydride; and wherein the pharmaceutically acceptable acid addition salt of rotigotine of formula I is derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, and tartaric acid.

5. (canceled)

6. The process of claim 4, wherein the acid addition salt of the compounds of formulae III and II is the hydrochloride salt; and wherein the pharmaceutically acceptable acid addition salt of rotigotine is the hydrochloride salt.

7. (canceled)

8. The process of claim 1, wherein the reaction in step-(c) is carried out at a temperature of about 0° C. to the reflux temperature of the solvent; wherein the Lewis acid used in step-(c) is selected from the group consisting of aluminium chloride, calcium chloride and zinc chloride; wherein the Lewis acid in step-(c) is used in an amount of about 1 to 5 equivalents with respect to the (−)-(S)-5-methoxy-2-[N-n-propyl-N-2-(2-thienyl)ethyl amino]tetralin of formula II; and wherein the thiourea in step-(c) is used in an amount of about 1 to 4 equivalents with respect to the (−)-(S)-5-methoxy-2-[N-n-propyl-N-2-(2-thienyl)ethylamino]tetralin of formula II.

9. The process of claim 8, wherein the reaction is carried out at a temperature of about 45° C. to about 100° C.; wherein the Lewis acid is aluminium chloride; wherein the Lewis acid is used in an amount of about 3 to 4 equivalents with respect to the (−)-(S)-5-methoxy-2-[N-n-propyl-N-2-(2-thienyl)ethylamino]tetralin of formula II; and wherein the thiourea is used in an amount of about 2.5 to 3.5 equivalents with respect to the (−)-(S)-5-methoxy-2-[N-n-propyl-N-2-(2-thienyl)ethylamino]tetralin of formula II.

10. (canceled)

11. (canceled)

12. (canceled)

13. (canceled)

14. A process for the preparation of (−)-(S)-5-hydroxy-2-[N-n-propyl-N-2-(2-thienyl)ethyl amino]tetralin (Rotigotine) of formula I: 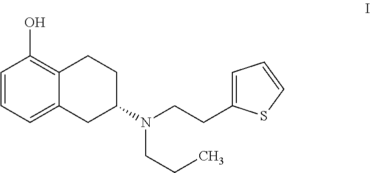
or a pharmaceutically acceptable salt thereof, comprising demethylating the compound of formula II: 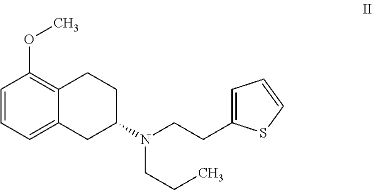
or an acid addition salt thereof, with a Lewis acid in the presence of thiourea in an organic solvent to produce rotigotine of formula I or a pharmaceutically acceptable salt thereof.

15. The process of claim 14, wherein the organic solvent is selected from the group consisting of n-pentane, n-hexane, n-heptane, cyclohexane, toluene, xylene, dichloromethane, ethylene dichloride, and mixtures thereof; wherein the Lewis acid is selected from the group consisting of aluminium chloride, calcium chloride and zinc chloride; and wherein the reaction is carried out at a temperature of about 0° C. to the reflux temperature of the solvent.

16. The process of claim 15, wherein the organic solvent is selected from the group consisting of n-pentane, n-hexane, n-heptane, cyclohexane, toluene, xylene, and mixtures thereof; wherein the Lewis acid is aluminium chloride; and wherein the reaction is carried out at a temperature of about 45° C. to about 100° C.

17. A process for purifying rotigotine hydrochloride, comprising:
a) providing a solution of crude rotigotine hydrochloride in a first solvent or in a solvent medium comprising the first solvent and a second solvent, wherein the first solvent is selected from the group consisting of N,N-dimethylacetamide, N,N-dimethylformamide, acetone, and mixtures thereof, and wherein the second solvent is selected from the group consisting of water, methanol, isopropanol, and mixtures thereof.
b) optionally, subjecting the solvent solution to carbon treatment or silica gel treatment; and
c) isolating the highly pure rotigotine hydrochloride from the solution and optionally converting the rotigotine hydrochloride obtained into highly pure rotigotine free base.

18. (canceled)

19. (canceled)

20. (canceled)

21. (canceled)

22. (canceled)

23. (canceled)

24. (canceled)

25. A process for preparing highly pure rotigotine or a pharmaceutically acceptable salt thereof comprising one, or more, of the ‘0.79 RRt’, ‘0.92 RRt’, ‘1.12 RRt’, ‘1.51 RRt’, ‘1.59 RRt’, ‘1.64 RRt’ and ‘1.79 RRt’ impurities each in an amount of about 0.01 area-% to about 0.1 area-% as measured by HPLC, comprising:
a) providing a first solution of crude rotigotine free base in a first solvent, wherein the first solvent is a chlorinated hydrocarbon solvent or a solvent medium comprising a chlorinated hydrocarbon solvent and an ester solvent;
b) subjected the first solution to silica gel treatment to provide a second solution; and
c) substantially removing the solvent from the second solution to produce an oily mass containing rotigotine free base;
d) combining the oily mass obtained in step-(c) with a second solvent to produce a third solution, and
e) isolating the highly pure rotigotine free base substantially free of impurities from the third solution, and optionally converting the highly pure rotigotine free base obtained into a pharmaceutically acceptable salt thereof.

26. The process of claim 25, wherein the first solvent used in step-(a) is selected from the group consisting of dichloromethane, ethylene dichloride, chloroform, carbon tetrachloride, ethyl acetate, methyl acetate, isopropyl acetate, tert-butyl methyl acetate, ethyl formate, and mixtures thereof; and wherein the second solvent used in step-(d) is selected from the group consisting of diisopropyl ether, diethyl ether, tetrahydrofuran, dioxane, n-pentane, n-hexane, n-heptane and their isomers, cyclohexane, and mixtures thereof.

27. The process of claim 26, wherein the first solvent is dichloromethane or a mixture of dichloromethane and ethyl acetate; and wherein the second solvent is n-heptane.

28. The process of claim 25, wherein the solution in step-(a) is provided either by dissolving or extracting the crude rotigotine free base in the first solvent at a temperature of above about 25° C., or by treating an acid addition salt of rotigotine with a base to produce rotigotine free base followed by extracting or dissolving the rotigotine free base in the first solvent at a temperature of above about 25° C.

29. The process of claim 28, wherein the rotigotine free base is dissolved or extracted in the first solvent at a temperature of about 25° C. to about 110° C.

30. The process of claim 25, wherein the first solution obtained in step-(a) is optionally stirred at a temperature of about 25° C. to about 110° C. for about 15 minutes to about 8 hours; wherein the silica gel treatment in step-(b) is carried out by stirring the solution with silica gel, by column chromatography, passing the solution through silica bed, or by circulation through silica cartridge repetitively; wherein the removal of solvent in step-(c) is accomplished by substantially complete evaporation of the solvent, concentrating the solution, or distillation of solvent under inert atmosphere, or a combination thereof; and wherein the isolation of highly pure rotigotine free substantially free of impurities in step-(e) is carried out by cooling, seeding, partial removal of the solvent from the solution, by adding an anti-solvent to the solution, or a combination thereof.

31. The process of claim 30, wherein the silica gel treatment in step-(b) is carried out by stirring the solution with silica gel at a temperature of below about 70° C. for at least 15 minutes followed by filtering the resulting mixture through a filtration bed to obtain the second solution containing rotigotine free base by removing silica gel; and wherein the crystallization in step-(e) is carried out by cooling the solution while stirring at a temperature of about 0° C. to about 25° C. for about 30 minutes to about 20 hours.

32. The process of claim 25, wherein the highly pure rotigotine free base substantially free of impurities obtained in step-(e) is recovered by filtration, filtration under vacuum, decantation, centrifugation, filtration employing a filtration media of a silica gel or celite, or a combination thereof; and wherein the pure rotigotine obtained is further dried under vacuum or at atmospheric pressure, at a temperature of about 35° C. to about 70° C.

33. (canceled)

34. (canceled)

35. (canceled)

36. (canceled)

37. (canceled)

38. (canceled)
